(19) United States
(12) Patent Application Publication
(10) Pub. No.: US 2012/0089136 A1
(43) Pub. Date: Apr. 12, 2012

(54) CRYOSURGICAL INSTRUMENT WITH QUICK COUPLING MECHANISM

(76) Inventors: Alexander Levin, Binyamina (IL); Didier Toubia, Ra'anana (IL); Miron Kaganovich, Haifa (IL)

(21) Appl. No.: 12/673,506

(22) PCT Filed: Aug. 13, 2008
(86) PCT No.: PCT/IL08/01114

§ 371 Date: Oct. 24, 2011

Related U.S. Application Data
(60) Provisional application No. 60/955,416, filed on Aug. 13, 2007.

(51) Int. Cl. A61B 18/02

(52) U.S. Cl. 606/23; 606/22

(57) ABSTRACT

A cryoprobe with a reliable and inexpensive quick coupling unit including a male unit and a suitable corresponding female coupling unit. The female unit is installed on the distal end of a hose supplying the liquid cryogen into the cryoprobe.

### FIELD OF THE INVENTION

The present invention relates to the area of cryosurgical equipment, and, specifically, to cryoprobes intended to be inserted in tissue or to be brought in contact with the tissue in order to perform a cryosurgical treatment such as cryoablation, cryo-analgesia or cryoadhesion.

### BACKGROUND OF THE INVENTION

There are some US patents and patent applications which describe designs of cryoprobes for internal and dermatological cryosurgical treatment. For example, U.S. Pat. No. 4,211,231 describes a cryosurgical instrument, which may be used with interchangeable spray and closed end cryotips includes a body member with a central bore extending partially therethrough. Refrigerant is supplied to a first point in the bore through a valved refrigerant supply line while an effluent opening connects an effluent line to a second point in the bore. The bore receives the inner ends of cryotips. All cryotips used with the instrument include refrigerant delivery tubes having inner ends which are positioned adjacent the first point in the bore and O-rings which are seated between the first point and the second point in the bore to prevent escape of refrigerant. Closed end cryotips to be used with the instrument further include an effluent channel extending from an expansion chamber to the second point in the bore. A second O-ring is provided with closed end cryotips. The second O-ring is seated between the second point in the bore and its open end to prevent escape of effluent refrigerant flowing through the effluent channel.

U.S. Pat. No. 6,468,269 describes a cryogenic system which is provided for performing cryosurgical interventions in the area of human and veterinary medicine, especially for treating tumors. The inventive system is comprised of a cryostat (a cryoprobe without its coupling unit) and a connecting device (the coupling unit), which is located between coaxial cryogenic lines for the direct flow and return flow of the cryogenic medium and which is provided for connecting a cryoinstrument to a cryo-attachment.

US patent application No. 20020160640 describes a connection assembly for conduits which are arranged coaxially inside one another and which accommodate the supply flow and return flow of a cryogenic medium. The assembly comprises a first and a second coupling half, which can be connected to one another in a sealed manner, whereby an inner conduit in each coupling half ends in a first conical sealing surface and an external conduit in each coupling half which encompasses the inner conduit ends in an additional conical sealing surface that is positioned outside the first conical sealing surface in the radial direction. In order to establish an electrical connection simply and reliably, at least one electrical contact is provided within the additional conical sealing surface in each coupling half.

However, these patents do not provide a cryoprobe with a reliable and inexpensive quick coupling unit at it proximal end, nor do they feature simple and sufficiently effective thermal insulation.

### SUMMARY OF THE INVENTION

The present invention provides, in some embodiments, a cryoprobe with a reliable and inexpensive quick coupling unit of the male type, as well, in some embodiments, a suitable corresponding female coupling unit, which is installed on the distal end of a hose supplying the liquid cryogen into the cryoprobe.

In an embodiment of this invention, a cryoprobe comprises a hollow shaft, which is provided at its distal end with a cryotip. The shaft is preferably fabricated from a rigid material. A proximal section of the shaft has smaller outer diameter than the rest of the shaft; this is obtained by turning-along. A supporting washer and an external bushing are installed on the proximal turned-along section of the shaft; a proximal face plane of the external bushing is locked by flanging the proximal end of the external shaft. The first bushing is optionally and preferably fabricated from an elastic material like rubber or some other synthetic flexible material.

A central feeding pipe is situated in the shaft; the proximal end of the central feeding pipe protrudes somewhat from the proximal end of the shaft. A proximal section of the central feeding pipe is provided with an outer annular groove fabricated by turning-along.

The internal surface of the shaft is provided with an annular groove opposite the outer annular groove of the central feeding pipe. These two annular grooves permit the shaft and the central feeding pipe to be joined by an omega shaped flat spring; at least the lower sections of the omega shaped flat spring are positioned in the annular groove of the shaft, and the middle sections of the omega shaped flat spring are positioned in the outer annular groove of the central feeding pipe. So positioned, the omega shaped flat spring plays two roles: it prevents relative displacement of the shaft and the central feeding pipe, on one hand, and centers the central feeding pipe relative to the shaft, on the other hand.

The extreme proximal section of the central feeding pipe is turned-along and a second bushing from a cryogenically stable material is installed on this extreme proximal section; Teflon or some other cryogenically stable polymers as are known in the art may optionally be used for fabrication of this second bushing.

The proximal end of the central feeding pipe is provided with an o-ring and flanged for hermetically sealing the second bushing.

Thermal insulation of the shaft is ensured by an intermediate tube with two flanged ends, wherein the outer diameter of the formed flanges conforms to the internal diameter of the shaft. Friction between the internal surface of the shaft and the flanges ensures stable positioning of the intermediate tube relative to the shaft.

Thermal insulation of the shaft can be provided by an external tube with two flanges directed inwards, wherein the inner diameter of these flanges conforms to the outer diameter of the shaft, and immobilization of the external tube with regard to the shaft is provided by friction between the flanges and the outer surface of the shaft, or by an additional immobilization means (for example, a screw, which fixes the position of the external tube regarding the shaft).

The proximal sections of the shaft and the central feeding pipe with their first and second bushings together form a male unit of the quick coupling of the cryoprobe.

A female unit of this quick coupling mechanism, which is in fluid communication with a hose serving to supply a liquid cryogen, comprises a cylindrical housing with an opening in its wall for removal of evaporated cryogen.

A face plane of this cylindrical housing is provided with a central opening for delivery of the liquid cryogen; the shape of the internal surface around the central opening ensures tight contact with the second bushing of the central feeding pipe. In addition, there is preferably a spring-actuated metal ring, which can be displaced along the internal wall of the cylindrical housing. Optionally the ring may be constructed of a rigid material.

This metal spring-actuated ring ensures tight contact with the external bushing of the shaft.

A spring-actuated ratchet is installed on the outer surface of the cylindrical housing and serves for locking the coupling consisting of the aforementioned male-female units.

Another embodiment of the cryoprobe comprises a hollow shaft, provided at its distal edge with a cryotip. The shaft is preferably fabricated from a rigid material.

A central feeding pipe is situated in the shaft; the proximal end of the central feeding pipe protrudes somewhat from the proximal end of the shaft. The extreme proximal section of the central feeding pipe is turned-along. The proximal sections of the shaft and the central feeding pipe serve for installation a male unit of a quick coupling. Thermal insulation of the shaft is ensured by an intermediate tube with two flanged ends, wherein the outer diameters of the formed flanges conform to the internal diameter of the shaft. Friction between the internal surface of shaft and the proximal flanging ensures stable positioning of the intermediate tube relative to the shaft.

Another optional embodiment for installation of the intermediate tube and shaft is based on forming two annular hollows in the shaft; these annular hollows preferably have an internal diameter corresponding to the outer diameter of the intermediate tube. In such a way, friction between the annular hollows and the intermediate tube ensures stable positioning of the intermediate tube in the internal cavity of the shaft.

Thermal insulation of the shaft can be provided by an external tube with two flanges directed inwards, wherein the inner diameter of these flanges conforms to the outer diameter of the shaft, while immobilization of the external tube relative to the shaft is provided by friction between the flanges and the outer surface of the shaft, or by an additional immobilization means (for example, a screw, which fixes the position of the external tube regarding the shaft).

The male unit of the quick coupling, which is installed on the proximal sections of the shaft and the central feeding pipe, comprises a bushing; the outer and internal surfaces of the bushing preferably have stepped cylindrical forms.

The outer surface of the bushing comprises proximal and distal cylindrical sections; the outer diameter of the distal section is somewhat larger than the outer diameter of the proximal section.

In another embodiment, the outer surface of the bushing is divided into three sections: the extreme distal and proximal sections have an equal diameter, while the middle section has a reduced diameter. The extreme distal and proximal outer sections are provided with circular grooves serving for installation of sealing o-rings.

In another embodiment, the grooves with their sealing O-rings are situated in a bushing of the female unit of the quick coupling, as described in greater detail below.

The inner surface of the bushing is preferably also stepped shaped, having distal, intermediate and proximal sections with progressively decreasing diameters.

The proximal and distal outer sections of the bushing are preferably provided with annular grooves, which serve for installation of o-rings; these o-rings are optionally and preferably fabricated from a cryogenically stable polymer, for example, Teflon or any other such polymer as is known in the art.

The bushing is preferably installed on the proximal sections of the shaft and the central feeding pipe in such a manner that the distal section of the inner surface of the bushing is fitted tightly on the proximal section of the shaft, while the proximal inner surface of the bushing is fitted slidingly on the turned-along proximal section of the central feeding pipe. After positioning the bushing on the proximal section of the shaft, the proximal edge of the central feeding pipe is preferably flanged with application of a deformable o-ring from a cryogenically stable polymer for sealing the gap between the proximal sections of the internal surface of the bushing and the central feeding pipe. Preferably a first through channel communicates between the internal and external spaces of the bushing between its inner intermediate section and its outer proximal section.

In the aforementioned optional embodiment, when the outer surface of the bushing is divided into three sections, the first through channel communicates between the internal and external spaces of the bushing between its inner intermediate section and its outer middle section.

A female unit of the quick coupling features a housing, having a stepped cylindrical inner cavity, wherein the diameter of the distal section of the inner cavity conforms to the outer diameter of the distal section of the bushing and the diameter of the middle section of the inner cavity conforms to the outer diameter of the proximal section of the bushing. It should be noted that the tolerances of these diameters allow sliding insertion of the bushing of the cryoprobe into the housing of the female unit, while the polymer o-rings, which are preferably installed in the aforementioned annular grooves of the bushing, ensure required sealing.

At the same time, these sections form an annular channel between the surfaces of the proximal section of the bushing and the distal section of the housing. A second through channel in the housing communicates between this annular channel and an outlet connector installed on the outer end of the second through channel.

In this way, the first through channel, annular channel and second through channel serve for exhausting evaporated cryogen from the cryoprobe.

For embodiments having the bushing with the outer surface divided into three sections, the extreme distal and proximal sections having an equal diameter, and the middle section having a reduced diameter, the internal cavity of the housing preferably has a simple cylindrical shape and the second through channel in the housing communicates with the annular channel formed between the middle outer section of the bushing and the internal cylindrical surface of the housing's cavity; an outlet connector is preferably installed on the outer end of the second through channel.

The face plane of the housing is provided with a central opening with an inlet connection installed on the outer end of this opening; this inlet connection serves for supplying the liquid cryogen into the cryoprobe.

In addition, the inner surface of the face plane of the housing preferably features a plurality of blind holes and helical springs, which are partially situated in these blind holes. In the process of coupling, the male unit of the coupling pair is therefore spring-actuated by these helical springs. Preferably also a spring-actuated ratchet is installed on the outer surface of the housing and serves for locking the quick coupling consisting of the aforementioned male-female units.

The coupling units, which enable mechanically joining the respective male to the respective female units, may optionally also feature one or more elements of electrical coupling, in order to permit electrical power to be supplied to the shaft or other elements of the probe, preferably for powering an electrical heating element or elements. In addition, this electrical coupling may provide power to thermal sensors arranged in the internal space of the cryosurgical instrument. In this case, the external shaft of the cryoprobe is optionally and preferably provided with a spiral-shaped electric heating element, for adjusting temperature on the internal side of the external shaft and/or with a thermal sensor such as a thermocouple.

Preferably an additional bushing, optionally and more preferably constructed from electro-isolating or insulating material, is installed on the external shaft of the cryoprobe distally to the bushing of the male unit of the coupling pair (as described in greater detail below). This additional bushing is preferably provided with one or more opening(s) for receiving one or more electrical wires to be connected to an electric heating element installed on the shaft, which more preferably features a helical structure. The electrical wires are then connected with electrical contacts installed on the additional bushing. The opposite electrical contacts, which are connected with a power supply unit, are installed in an additional female bushing. This additional female bushing is fastened distally to the housing of the female unit of the coupling and is optionally and preferably constructed from electro-isolating or insulating material as well.

### BRIEF DESCRIPTION OF THE DRAWINGS

FIG. 1a shows an axial cross-section of a cryoprobe with application of a flat spring as a joining element of the cryoprobe construction.

FIG. 1b is a transversal cross-section A-A of the cryoprobe with application of a flat spring as a joining element of the cryoprobe construction.

FIG. 2a is an axial cross-section of a cryoprobe with application of a bushing as a joining element of the cryoprobe construction and a female unit of the quick coupling.

FIG. 2b is an axial cross-section of a cryoprobe with application of a bushing as a joining element of the cryoprobe construction.

FIG. 2c is an axial cross-section the female unit of the quick coupling presented in FIG. 2a.

FIG. 3 shows an axial cross-section of a cryoprobe with application of a flat spring as a joining element of the cryoprobe construction and with thermal insulation by an external tube.

FIG. 4 is an axial cross-section of a cryoprobe with application of a bushing as a joining element of the cryoprobe construction and with thermal insulation by an external tube.

FIG. 5a is an axial cross-section of a cryoprobe with application of a bushing as a joining element of the cryoprobe construction and a female unit of the quick coupling, wherein the outer surface of the bushing is divided into three sections: the extreme distal and proximal sections having an equal diameter, and the middle section having a reduced diameter.

FIG. 5b is an axial cross-section of a cryoprobe with application of a bushing as a joining element of the cryoprobe construction and the outer surface of the bushing is divided into three sections: the extreme distal and proximal sections have an equal diameter, and the middle section has a reduced diameter.

FIG. 5c is an axial cross-section the female unit of the quick coupling presented in FIG. 5a.

FIG. 6 is an axial cross-section of a cryoprobe with application of a bushing as a joining element of the cryoprobe construction and annular hollows of the shaft as fixing elements for the intermediate tube.

FIG. 7a is an axial cross-section of a cryoprobe with application of a bushing as a joining element of the cryoprobe construction and a female unit of the quick coupling, wherein sealing O-rings are installed constantly in the female unit of the quick coupling.

FIG. 7b is an axial cross-section of a cryoprobe with application of a bushing as a joining element of the cryoprobe construction as it is shown in FIG. 7a.

FIG. 7c is an axial cross-section the female unit of the quick coupling presented in FIG. 7a.

FIG. 8 is an axial cross-section of an optional embodiment of a cryoprobe constructed as shown in FIG. 7a, 7b and FIG. 7c.

FIG. 9 is an axial cross-section of another optional embodiment of a cryoprobe constructed as shown in FIG. 7a, 7b and FIG. 7c.

FIG. 10 is an axial cross-section of an optional embodiment of a cryoprobe as shown in FIG. 7a, wherein the female unit is provided with a distal annular groove with an additional O-ring installed in it, and a circular groove, which serves for drainage of a liquid cryogenic fluid.

FIG. 11 is an axial cross-section of another optional embodiment of a cryoprobe constructed as shown in FIG. 8, where a thermocouple is situated adjacently to the external shaft.

### DESCRIPTION OF PREFERABLE EMBODIMENTS

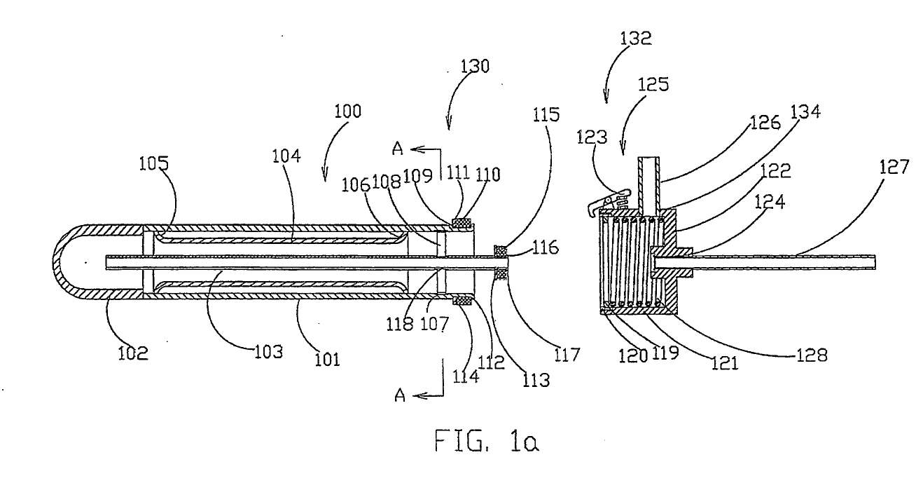

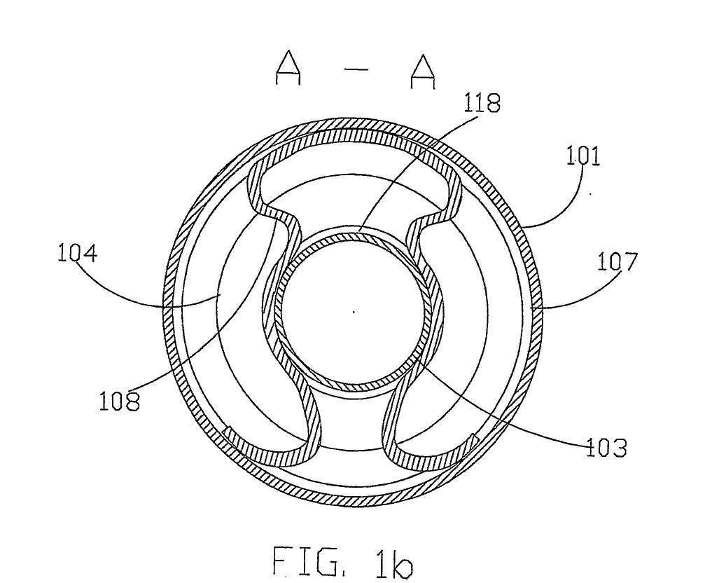

FIG. 1a and FIG. 1b show an axial cross-section and a transversal cross-section A-A of exemplary embodiments of a cryoprobe having a flat spring as a joining element of the cryoprobe construction.

Cryoprobe 100 comprises hollow shaft 101, which is joined at its distal edge with cryotip 102. Shaft 101 is preferably fabricated from a rigid material. The proximal outer section 109 of shaft 101 has a smaller outer diameter formed, for example, through turning-along, in which the diameter is reduced continually. A supporting washer 111 and an external bushing 110 are installed on an outer surface of the proximal outer section 109 of shaft 101; the proximal face plane of the external bushing is locked by flanging 112 extending outwardly from the proximal edge of shaft 101. The external bushing 110 is optionally and preferably fabricated from an elastic material like rubber or a flexible synthetic material.

A central feeding pipe 103 is situated in shaft 101; the proximal end of the central feeding pipe 101 protrudes somewhat from the proximal end of shaft 101. The proximal outer section of the central feeding pipe is provided with an outer annular groove 118 fabricated, for example, by turning-along.

The internal surface of the shaft is provided with an annular groove 107 opposite the outer annular groove 118 of the central feeding pipe 103, for joining shaft 101 and the central feeding pipe 103 with an omega shaped flat spring 108; at least the lower sections of the omega shaped flat spring 108 are positioned in the annular groove 107 of shaft 101, and the middle sections of the omega shaped flat spring are positioned in the outer annular groove 118 of the central feeding pipe 103. In this way, the omega shaped flat spring 108 plays two roles: it prevents relative displacement of shaft 101 and the central feeding pipe 103 on one hand, and it centers the central fee ding pipe 103 with regard to shaft 101 on the other hand.

The extreme proximal section 113 of the central feeding pipe 103 is turned-along as well and a second bushing 115 from a cryogenically stable material with a second supporting washer are preferably installed on this extreme proximal section 113. Teflon or some other cryogenically stable polymers are preferably used for fabrication of this bushing as is known in the art.

The proximal end of the central feeding pipe 103 is preferably provided with o-ring 116 and outwardly extending flanging 117 for hermetically sealing the second bushing.

Thermal insulation of the shaft 101 is ensured by an intermediate tube 104 with two flanged ends 105 and 106, wherein the outer diameters of the formed flanges 105 and 106 conform to the internal diameter of the shaft 101. Friction between the internal surface of shaft 101 and flanging 105, 106 ensures stable positioning of the intermediate tube 104 relative to shaft 101.

In combination, the proximal sections of shaft 101 and the central feeding pipe 103 with their bushings 115 and 110 comprise a male unit 130 of the quick coupling mechanism 132 of the cryoprobe 100.

A female unit 125 of this quick coupling mechanism is preferably in fluid communication with a hose 127 for supplying a fluid cryogen, e.g. liquid cryogen, and preferably comprises a cylindrical housing 121 with an outlet connection 126 in its wall 134 for removal of evaporated cryogen.

A face plane 122 of this housing is provided with a central opening 124 for delivery of the liquid cryogen; the form of the internal surface around the central opening 124 ensures tight contact with the second bushing 115 of the central feeding pipe 103. In addition, there is preferably a spring-actuated metal ring 119, which can be displaced along the internal wall of the cylindrical housing 121. Ring 119 may optionally be made of a rigid material. Distal displacement of this spring-actuated metal ring is restricted by a distal skirting 120. A helical spring 128 serves for spring-actuating the metal ring 119. This metal spring-actuated ring 119 ensures tight contact with the external bushing 110 of shaft 101.

Preferably a spring-actuated ratchet 123 is installed on the outer surface of the cylindrical housing 121 and locks the coupling consisting of the aforementioned male-female units 130, 125.

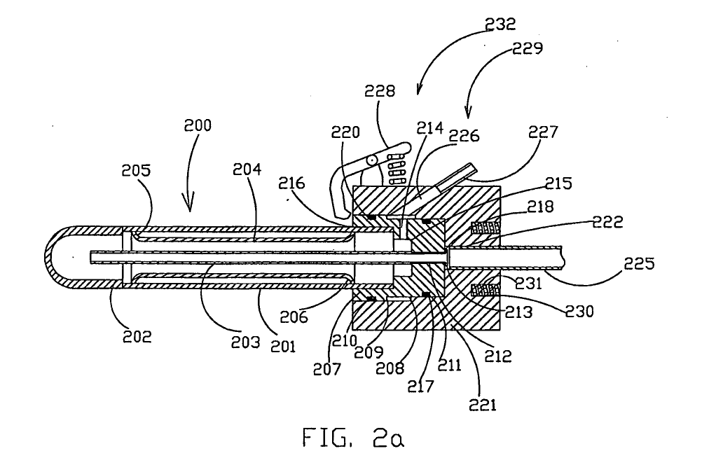

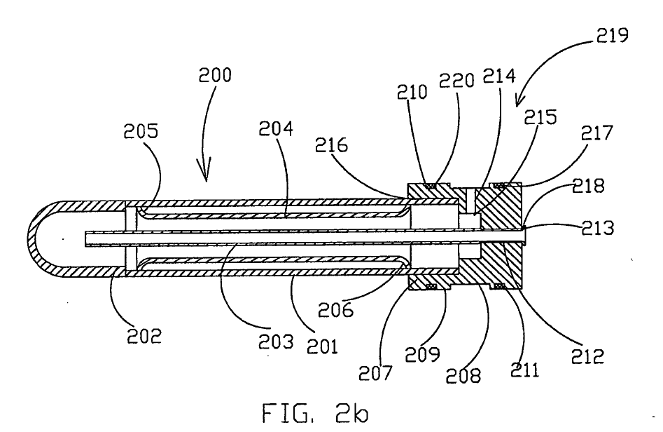

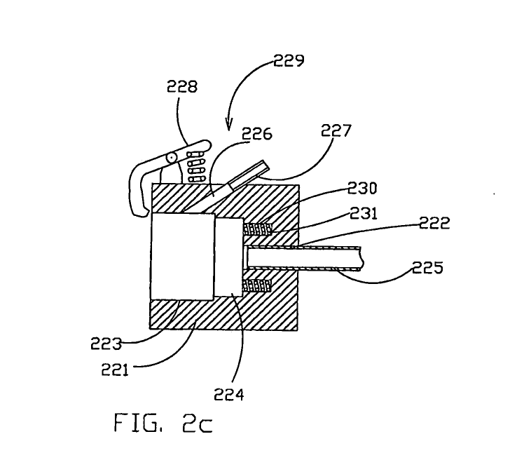

FIG. 2a, FIG. 2b and FIG. 2c show an axial cross-section of an exemplary embodiment of a cryoprobe 200.

Cryoprobe 200 comprises a hollow shaft 201, provided at its distal end with cryotip 202. Shaft 201 is preferably fabricated from a rigid material.

A central feeding pipe 203 is situated in shaft 201; the proximal end of the central feeding pipe 203 protrudes somewhat from the proximal end of shaft 201. The extreme proximal section of the central feeding pipe 203 is turned-along. The proximal sections of shaft 201 and the central feeding pipe 203 serve for installation of a male unit 219 of a quick coupling mechanism 232.

Thermal insulation of shaft 201 is ensured by an intermediate tube 204 with two flanged ends 205 and 206, wherein the outer diameter of the formed flanges 205 and 206 conforms to the internal diameter of the shaft. Friction between the internal surface of shaft 201 and flanging 205, 206 ensures stable positioning of the intermediate tube 204 with regard to shaft 201.

The male unit 219 of the quick coupling mechanism, which is installed on the proximal sections of shaft 201 and the central feeding pipe 203, comprises bushing 207; the outer and internal surfaces of this bushing 207 are preferably stepped.

The outer surface of bushing 207 comprises proximal and distal cylindrical sections 208 and 209, respectively; the outer diameter of the distal section 209 is preferably somewhat larger than the outer diameter of the proximal section 208. The proximal and distal cylindrical sections 208 and 209 are preferably provided with annular grooves 211 and 210 for installation of o-rings 220 and 217, which are more preferably fabricated from cryogenically stable polymer.

The inner surface of bushing 207 is preferably also stepped, with distal, intermediate and proximal sections 216, 215 and 212, respectively, featuring progressively decreasing diameters.

Bushing 207 is installed on the proximal sections of shaft 201 and the central feeding pipe 203 in such a manner that the distal section of the inner surface of the bushing is fitted tightly to the proximal section of the shaft and the proximal inner surface 212 of bushing 207 is fitted slidingly on the turned-along proximal section of the central feeding pipe 203. After positioning bushing 207 on the proximal section of shaft 201, the proximal edge of the central feeding pipe 203 is flanged with application of a deformable o-ring 218, preferably from a cryogenically stable polymer, for sealing the gap between the proximal sections of the internal surface of bushing 207 and the central feeding pipe 203. A first through channel 214 communicates between the internal and external spaces of the bushing, between its inner intermediate section 215 and its outer proximal section 208.

A female unit 229 of the quick coupling mechanism features housing 221 having a stepped cylindrical inner cavity, wherein the diameter of the distal section 223 of the inner cavity conforms to the outer diameter of the distal section 209 of bushing 207 and the diameter of the middle section 224 of the inner cavity conforms to the outer diameter of the proximal section 208 of bushing 207. A proximal section 222 of the inner cavity serves for installation of an inlet connection 225 supplying the cryogen into cryoprobe 200. The tolerances of these diameters allow sliding insertion of bushing 207 of cryoprobe 200 into housing 221 of the female unit 229 and the polymer o-rings 220 and 217 installed in the aforementioned annular grooves of bushing 207 ensure required sealing.

In addition, the inner surface of the face plane of housing 221 is provided with blind holes 230 and helical springs 231, which are partially situated in these blind holes 230. In such a way, in the process of coupling, the male unit 219 of the coupling pair is spring-actuated by these helical springs 231.

At the same time, the lengths of the outer distal and proximal sections 209 and 208, respectively, of bushing 207 and the inner distal and middle sections 223 and 224 of housing 221 form an annular channel between the surfaces of the outer proximal section 208 of bushing 207 and the inner distal section 223 of housing 221.

A second through channel 226 with an outlet connection 227 installed on the outer end of the second through channel 226, communicates the aforementioned annular channel with a space outside of housing 221.

In such a way, these first through channel 214, annular channel and second through channel 226 serve for exhausting evaporated cryogen from cryoprobe 200.

A spring-actuated ratchet 228 is installed on the outer surface of the housing 221.

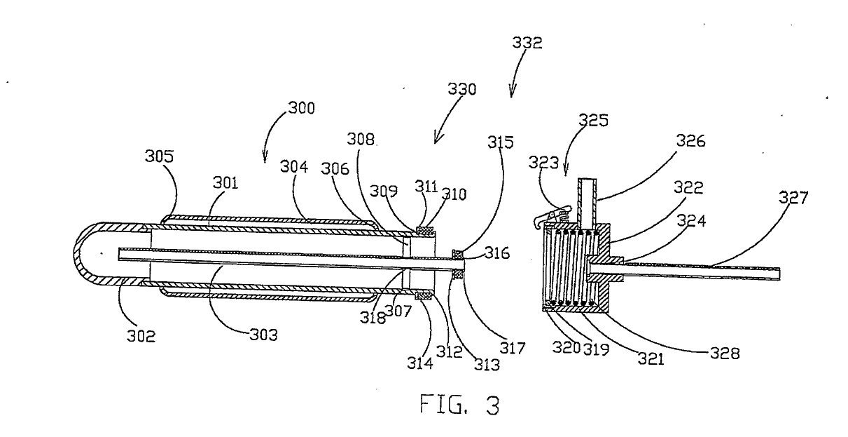

FIG. 3 shows an axial cross-section of a cryoprobe with application of a flat spring as a joining element of the cryoprobe construction and with thermal insulation of the shaft by an external tube.

Cryoprobe 300 comprises hollow shaft 301, which is joined at its distal edge with cryotip 302. Shaft 301 is preferably fabricated from a rigid material. The proximal outer section 309 of shaft 301 has smaller outer diameter; this is obtained by turning-along. A supporting washer 311 and an external bushing 310 are installed on the outer surface of the proximal outer section 309 of shaft 301; the proximal face plane of the external bushing is locked by flanging 312. The external bushing 310 is fabricated from an elastic material like rubber.

A central feeding pipe 303 is situated in shaft 301; the proximal end of the central feeding pipe 301 protrudes somewhat from the proximal end of shaft 301. The proximal outer section of the central feeding pipe is provided with an outer annular groove 318 fabricated by turning-along.

The internal surface of the shaft is provided with an annular groove 307 opposite the outer annular groove 318 of the central feeding pipe 303.

This allows to join shaft 301 and the central feeding pipe 303 by an omega shaped flat spring 308; at least the lower sections of the omega shaped flat spring 308 are positioned in the annular groove 307 of shaft 301, and the middle sections of the omega shaped flat spring are positioned in the outer annular groove 318 of the central feeding pipe 303.

So positioned, the omega shaped flat spring 308 plays two roles: it prevents relative displacement of shaft 301 and the central feeding pipe 303 on one hand, and it centers the central feeding pipe 303 relative to shaft 301 on the other hand.

The extreme proximal section 313 of the central feeding pipe 303 is turned-along too and a second bushing 315 from a cryogenically stable material with the second supporting washer 314 are installed on this extreme proximal section 313. Teflon or some other cryogenically stable polymers can be used for fabrication of this second bushing.

The proximal end of the central feeding pipe is provided with o-ring 316 and flanging 317; this allows locking hermetically the second bushing.

Thermal insulation of the shaft is ensured by an external tube 304 with two ends 305 and 306 flanged inwardly, wherein the inner diameters of the formed flanges 305 and 306 conform to the outer diameter of the shaft. Friction between the internal surface of shaft 301 and flanging 305, 306 ensures stable positioning the external tube 304 relative to shaft 301.

The proximal sections of shaft 301 and the central feeding pipe 303 with their bushings 315 and 310 together form a male unit 330 of a quick coupling mechanism 332 of the cryoprobe.

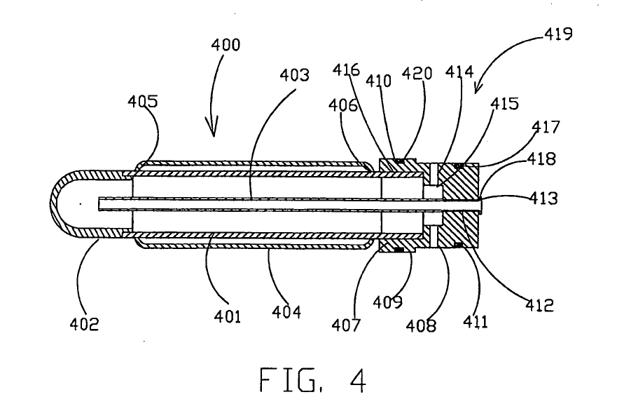

FIG. 4 is an axial cross-section of a cryoprobe with application of a bushing as a joining element of the cryoprobe construction and with thermal insulation by an external tube.

This embodiment of cryoprobe 400 comprises hollow shaft 401, which is joined at its distal edge with cryotip 402. Shaft 401 is preferably fabricated from a rigid material.

A central feeding pipe 403 is situated in shaft 403; the proximal end of the central feeding pipe 403 protrudes somewhat from the proximal end of shaft 401. The extreme proximal section of the central feeding pipe 403 is turned-along. The proximal sections of shaft 401 and the central feeding pipe 403 serve for installation a male unit 419 of a quick coupling.

Thermal insulation of shaft 401 is ensured by an external tube 404 with two ends 405 and 406 flanged inwardly, wherein the inner diameter of the formed flanges 405 and 406 conforms to the outer diameter of shaft 401. Friction between the inner surface of shaft 401 and flanging 406 ensures stable positioning the external tube 404 regarding shaft 401.

The male unit 419 of the quick coupling, which is installed on the proximal sections of shaft 401 and the central feeding pipe 403, comprises bushing 407, the outer and internal surface of this bushing 407 have a step-wise forms.

The outer surface of bushing 407 comprises proximal and distal cylindrical sections 408 and 409, respectively; the outer diameter of the distal section 409 is somewhat larger than the outer diameter of the proximal section 408. The proximal and distal cylindrical sections 408 and 409 are provided with annular grooves 411 and 410 for installation of cryogenically stable polymer o-rings 420 and 417.

The inner surface of bushing 407 is shaped too in the step-wise form: it has distal, intermediate and proximal sections 416, 415 and 412, respectively, with progressively decreasing diameters.

Bushing 407 is installed on the proximal sections of shaft 401 and the central feeding pipe 403 in such a manner, that the distal section of the inner surface of the bushing is fitted tightly on the proximal section of the shaft and the proximal inner surface 412 of bushing 407 is fitted slidingly on the turned-along proximal section of the central feeding pipe 403. After positioning bushing 407 on the proximal section of shaft 401, the proximal edge of the central feeding pipe is flanged with application of a deformable o-ring 418, fabricated from a cryogenically stable polymer, for sealing the gap between the proximal sections of the internal surface of bushing 407 and the central feeding pipe 403. A first through channel 414 communicates the internal and external spaces of the bushing in the place of its inner intermediate section 415 and its outer proximal section 408.

A female unit of the coupling is designed in this embodiment like it is shown in FIG. 2c.

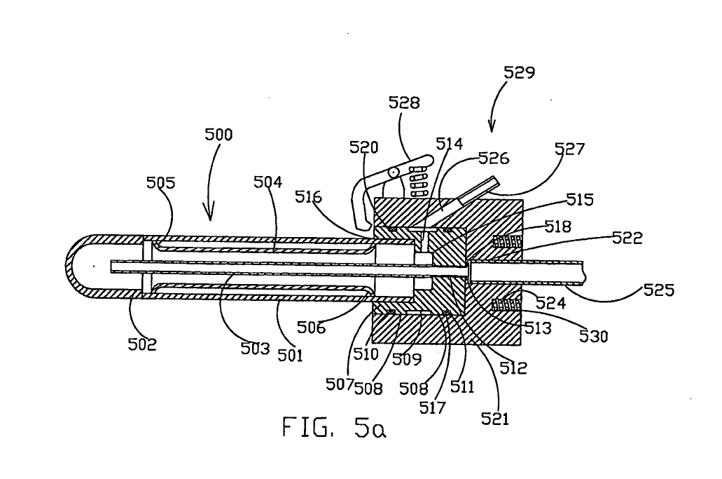

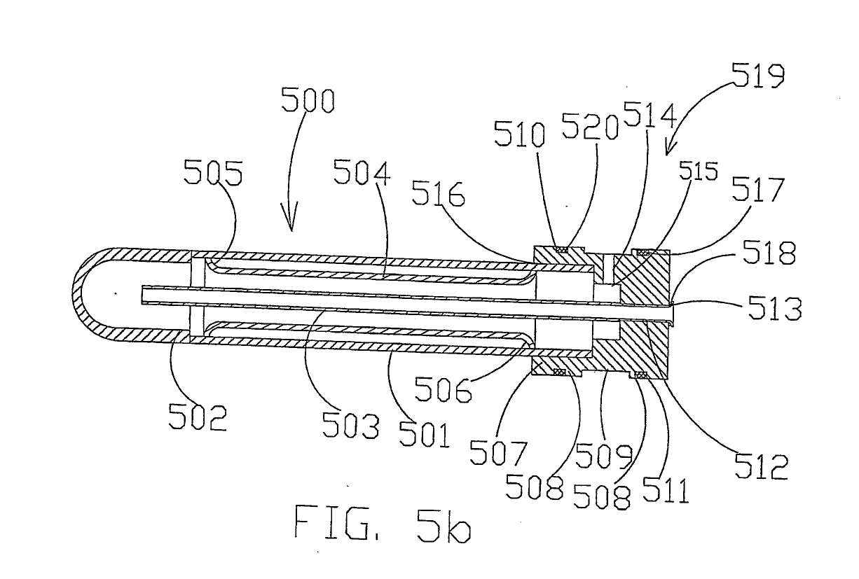

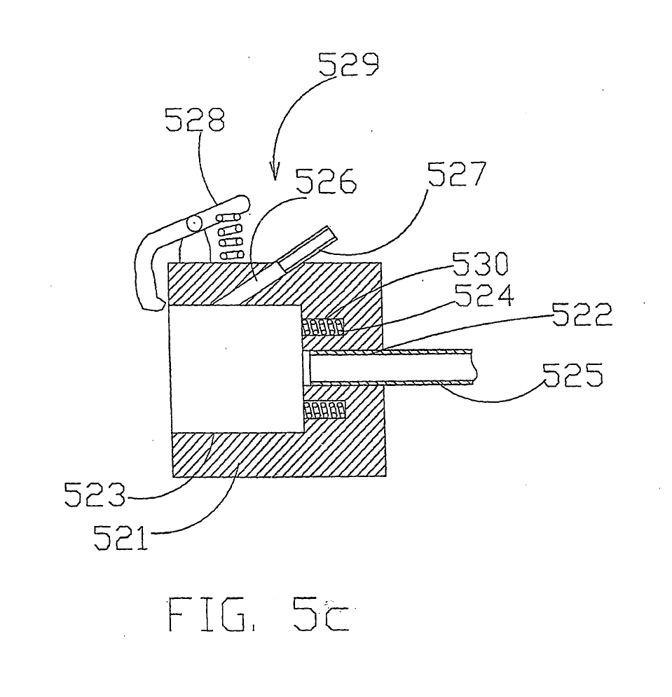

FIG. 5a, FIG. 5b and FIG. 5c show an axial cross-section of a cryoprobe with application of a bushing as a joining element of the cryoprobe construction and a female unit of the quick coupling, wherein the outer surface of the bushing is divided on three sections: the extreme distal and proximal sections having an equal diameter, and the middle section having a reduced diameter.

This embodiment of cryoprobe 500 comprises hollow shaft 501, which is provided at its distal end with cryotip 502. Shaft 501 is preferably fabricated from a rigid material.

A central feeding pipe 503 is situated in shaft 503; the proximal end of the central feeding pipe 503 protrudes somewhat from the proximal end of shaft 501. The extreme proximal section of the central feeding pipe 503 is turned-along. The proximal sections of shaft 501 and the central feeding pipe 503 serve for installation a male unit 519 of a quick coupling.

Thermal insulation of shaft 501 is ensured by an intermediate tube 504 with two flanged ends 505 and 506, wherein the outer diameter of the formed flanges 505 and 506 conforms to the internal diameter of the shaft. Friction between the internal surface of shaft 501 and flanging 505, 506 ensures stable positioning of the intermediate tube 504 relative to shaft 501.

The male unit 519 of the quick coupling, which is installed on the proximal sections of shaft 501 and the central feeding pipe 503, comprises bushing 507, the outer and internal surface of this bushing 507 have step-wise forms.

The outer surface of bushing 507 comprises proximal and distal cylindrical sections 508 and a middle section 509; the proximal and distal sections 508 have the same diameter, and diameter of the middle section 509 is somewhat smaller. The proximal and distal cylindrical sections 508 are provided with annular grooves 511 and 510 for installation of cryogenically stable polymer o-rings 520 and 517.

The inner surface of bushing 507 is shaped too in the step-wise form: it has distal, intermediate and proximal sections 516, 515 and 512, respectively, with progressively decreasing diameters.

Bushing 507 is installed on the proximal sections of shaft 501 and the central feeding pipe 503 in such a manner, that the distal section of the inner surface of the bushing is fitted tightly on the proximal section of the shaft and the proximal inner surface 512 of bushing 507 is fitted slidingly on the turned-along proximal section of the central feeding pipe 503. After positioning bushing 507 on the proximal section of shaft 501, the proximal edge of the central feeding pipe 503 is flanged with application of a deformable o-ring 518, fabricated from a cryogenically stable polymer, for sealing the gap between the proximal sections of the internal surface of bushing 507 and the central feeding pipe 503. A first through channel 514 communicates the internal and external spaces of bushing 507 in the place of its inner intermediate section 515 and its outer middle section 509.

A female unit 529 of the quick coupling comprises housing 521, with a cylindrical inner cavity, wherein the diameter of the cylindrical section 523 of the inner cavity conforms to the outer diameter of the distal and proximal sections 508 of bushing 507.

A through opening 522 in the proximal face plane of the inner cavity serves for installation of an inlet connection 525 supplying the cryogen into cryoprobe 500. It should be noted, that the tolerances of the bushing and the housing allow to insert slidingly bushing 507 of cryoprobe 500 into housing 521 of the female unit 529 and the polymer o-rings 520 and 517 installed in the aforementioned annular grooves 510 and 511 of bushing 507 ensure required sealing.

In addition, the inner surface of the face plane of housing 521 is provided with blind holes 530 and helical springs 524, which are partially situated in these blind holes 530. In such a way, in the process of coupling, the male unit 519 of the coupling pair is spring-actuated by these helical springs 524.

A second through channel 526 with an outlet connection 527 installed on the outer end of the second through channel 526, communicates the annular channel formed between the middle section 509 of bushing 507 and a space outside of housing 521.

In such a way, these first through channel 514, the annular channel and second through channel 526 serve for exhausting evaporated cryogen from cryoprobe 500.

A spring-actuated ratchet 528 is installed on the outer surface of the housing 521.

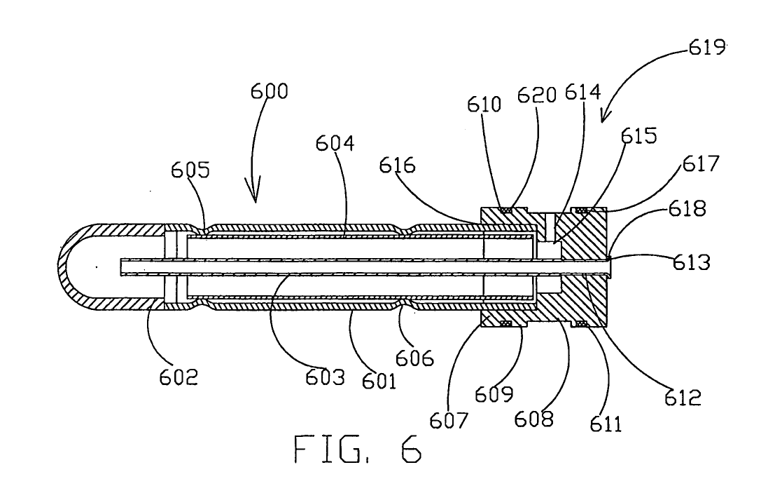

FIG. 6 shows an axial cross-section of a cryoprobe with application of a bushing as a joining element of the cryoprobe construction and annular hollows of the shaft as fixing elements for the intermediate tube. This embodiment of cryoprobe 600 comprises hollow shaft 601, which is provided at its distal end with cryotip 602. Shaft 601 is preferably fabricated from a rigid material.

A central feeding pipe 603 is situated in shaft 601; the proximal end of the central feeding pipe 203 protrudes somewhat from the proximal end of shaft 601. The extreme proximal section of the central feeding pipe 603 is turned-along. The proximal sections of shaft 601 and the central feeding pipe 603 serve for installation a male unit 619 of a quick coupling.

Thermal insulation of shaft 601 is ensured by an intermediate tube 604 and two annular hollows 605 and 606 in the shaft; these annular hollows have an internal diameter corresponding to the outer diameter of the intermediate tube 604. In such a way, friction between the annular hollows 605, 606 and the intermediate tube 604 ensures stable positioning of the intermediate tube 604 in the internal cavity of shaft 601.

The male unit 619 of the quick coupling, which is installed on the proximal sections of shaft 601 and the central feeding pipe 603, comprises bushing 607, the outer and internal surface of this bushing 607 have step-wise forms.

The outer surface of bushing 607 comprises proximal and distal cylindrical sections 609, and middle section 608; the outer diameter of the proximal and distal sections 609 is somewhat larger than the outer diameter of the middle section 608. The proximal and distal cylindrical sections 609 are provided with annular grooves 611 and 610 for installation of cryogenically stable polymer o-rings 620 and 617.

The inner surface of bushing 607 is shaped too in the step-wise form: it has distal, intermediate and proximal sections 616, 615 and 612, respectively, with progressively decreasing diameters.

Bushing 607 is installed on the proximal sections of shaft 601 and the central feeding pipe 603 in such a manner, that the distal section of the inner surface of the bushing is fitted tightly on the proximal section of the shaft and the proximal inner surface 612 of bushing 607 is fitted slidingly on the turned-along proximal section of the central feeding pipe 603. After positioning bushing 607 on the proximal section of shaft 601, the proximal edge of the central feeding pipe 603 is flanged with application of a deformable o-ring 618, fabricated from a cryogenically stable polymer, for sealing the gap between the proximal sections of the internal surface of bushing 607 and the central feeding pipe 603.

A first through channel 614 communicates the internal and external spaces of the bushing in the place of its inner intermediate section 615 and its outer proximal section 608.

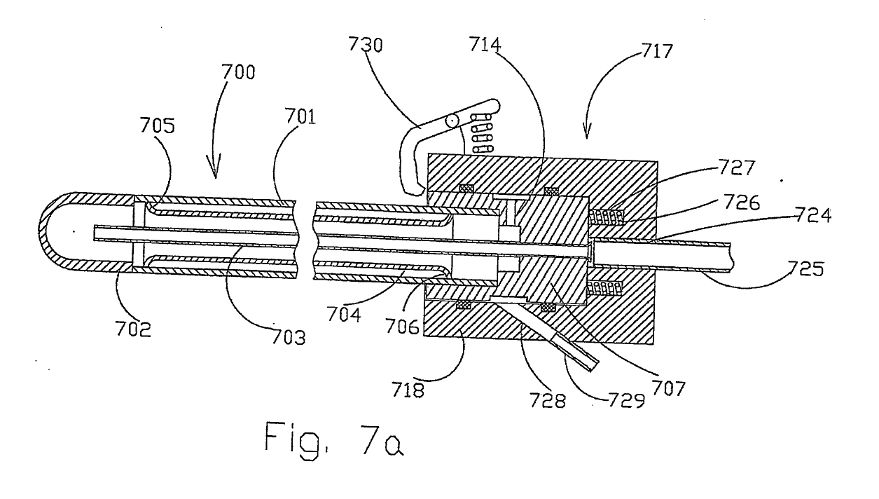

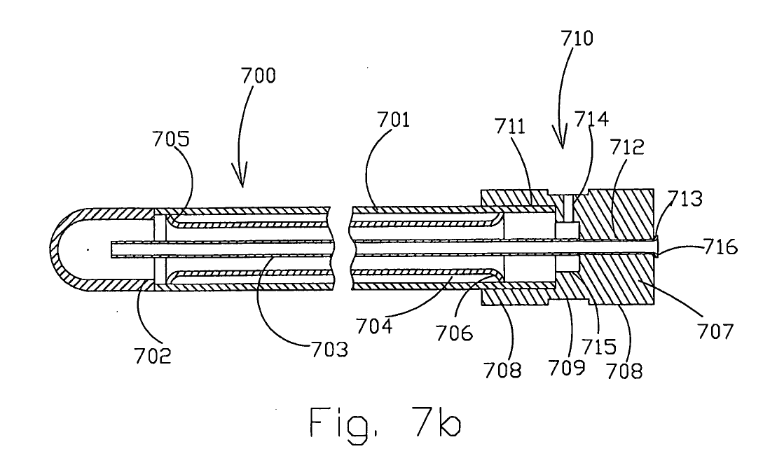

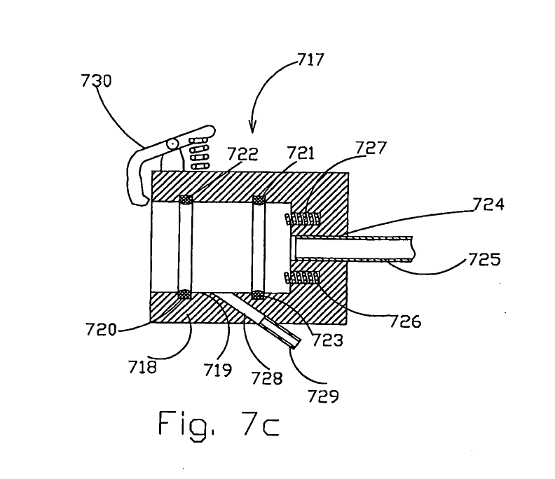

FIG. 7a, FIG. 7b and FIG. 7c show an axial cross-section of a cryoprobe with application of a bushing as a joining element of the cryoprobe construction and a female unit of the quick coupling, wherein sealing O-rings are installed stationary in a female unit of the quick coupling.

This embodiment of cryoprobe 700 comprises hollow shaft 701, which is provided at its distal end with cryotip 702. Shaft 701 is preferably fabricated from a rigid material.

A central feeding pipe 703 is situated in shaft 703; the proximal end of the central feeding pipe 703 protrudes somewhat from the proximal end of shaft 701. The extreme proximal section of the central feeding pipe 703 is turned-along. The proximal sections of shaft 701 and the central feeding pipe 703 serve for installation of a male unit 710 of a quick coupling.

Thermal insulation of shaft 701 is ensured by an intermediate tube 704 with two flanged ends 705 and 706, wherein the outer diameter of the formed flanges 705 and 706 conforms to the internal diameter of the shaft. Friction between the internal surface of shaft 701 and flanging 705, 706 ensures stable positioning of the intermediate tube 704 relative to shaft 701.

The male unit 710 of the quick coupling, which is installed on the proximal sections of shaft 701 and the central feeding pipe 703, comprises bushing 707.

The outer surface of bushing 707 comprises proximal and distal cylindrical sections 708 and a middle section 709; the proximal and distal sections 708 have the same diameter, and diameter of the middle section 709 is somewhat smaller.

The inner surface of bushing 707 is shaped too in the step-wise form: it has distal, intermediate and proximal sections 711, 715 and 712, respectively, with progressively decreasing diameters.

Bushing 707 is installed on the proximal sections of shaft 701 and the central feeding pipe 703 in such a manner, that the distal section of the inner surface of the bushing is fitted tightly on the proximal section of the shaft and the proximal inner surface 712 of bushing 707 is fitted slidingly on the turned-along proximal section of the central feeding pipe 703. After positioning bushing 707 on the proximal section of shaft 701, the proximal edge of the central feeding pipe 703 is flanged with formation flange 713 and application of a deformable o-ring 716, fabricated from a cryogenically stable polymer, for sealing the gap between the proximal sections of the internal surface of bushing 707 and the central feeding pipe 703. A first through channel 714 communicates the internal and external spaces of bushing 707 in the place of its inner intermediate section 715 and its outer middle section 709.

A female unit 717 of the quick coupling comprises housing 718, having a cylindrical inner cavity, wherein the diameter of the cylindrical section 719 of the inner cavity conforms to the outer diameter of the distal and proximal sections 708 of bushing 707.

The cylindrical section 719 is provided with distal and proximal annular grooves 720 and 721, which serve for installation of steady sealing O-rings 723 and 722.

A through opening 724 in the proximal face plane of the inner cavity serves for installation of an inlet connection 725 supplying the cryogen into cryoprobe 700. It should be noted, that the tolerances of the bushing and the housing allow to insert slidingly bushing 707 of cryoprobe 700 into housing 718 of the female unit 717 and the polymer o-rings 723 and 722 installed in the aforementioned annular grooves 720 and 721 of housing 718 ensure required sealing.

In addition, the inner surface of the face plane of housing 718 is provided with blind holes 726 and helical springs 727, which are partially situated in these blind holes 726. In such a way, in the process of coupling, the male unit 710 of the coupling pair is spring-actuated by these helical springs 727.

A second through channel 728 with an outlet connection 729 installed on the outer end of the second through channel 728, communicates the annular channel formed between the middle section 709 of bushing 707 with a space outside of housing 718.

In such a way, the first through channel 714, annular channel and second through channel 728 serve for exhausting evaporated cryogen from cryoprobe 700.

A spring-actuated ratchet 730 is installed on the outer surface of the housing 718.

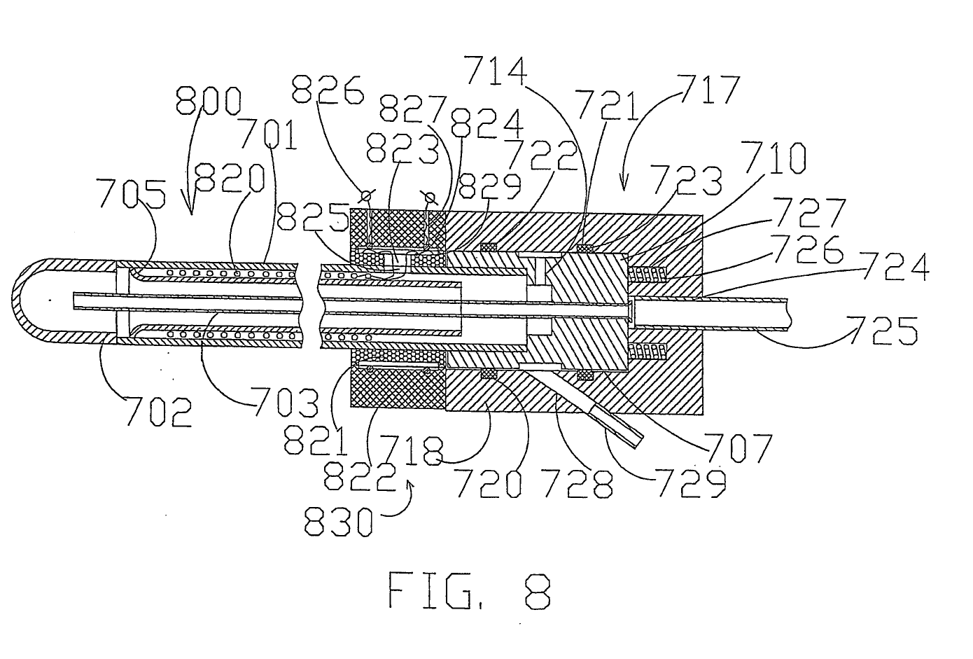

FIG. 8 is an axial cross-section of an optional embodiment of cryoprobe 800 constructed as shown in FIG. 7a, 7b and FIG. 7c; this cryoprobe is provided with an internal electric heating spiral and an electrical quick coupling unit. Reference numbers that are the same as for FIG. 7 have the same or similar function (however, the proximal flanging 706 and spring-actuated ratchet 730 are not featured and the inner surfaces of bushing 707: 711, 715, 712, the proximal, middle and distal cylindrical sections 708, 709 of outer surface of bushing 707 and the cylindrical section 719 of the female unit 717 are not shown).

This embodiment, featuring certain changes to cryoprobe 700 of FIG. 7, comprises hollow shaft 701, provided at its distal end with cryotip 702. Shaft 701 is preferably fabricated from a rigid material.

A central feeding pipe 703 is situated in shaft 703. The proximal end of the central feeding pipe 703 protrudes somewhat from the proximal end of shaft 701. The extreme proximal section of the central feeding pipe 703 is turned-along. The proximal sections of shaft 701 and the central feeding pipe 703 serve for installation of a male unit 710 of a quick coupling mechanism.

Thermal insulation of shaft 701 is ensured by an intermediate tube 704 with a flanged end 705, wherein the outer diameter of the formed flange 705 conforms to the internal diameter of the shaft. Friction between the internal surface of shaft 701 and flanging 705 ensures stable positioning of the intermediate tube 704 relative to shaft 701.

The male unit 710 of the quick coupling, which is installed on the proximal sections of shaft 701 and the central feeding pipe 703, comprises bushing 707. The outer surface of bushing 707 comprises proximal and distal cylindrical sections 708 and a middle section 709; the proximal and distal sections 708 have the same diameter, and diameter of the middle section 709 is preferably somewhat smaller.

The inner surface of bushing 707 preferably also has a stepped shape: it has distal, intermediate and proximal sections 711, 715 and 712, respectively, which preferably feature progressively decreasing diameters.

Bushing 707 is installed on the proximal sections of shaft 701 and the central feeding pipe 703 in such a manner that the distal section of the inner surface of the bushing is tightly fitted to the proximal section of the shaft and the proximal inner surface 712 of bushing 707 slidingly fits along on the turned-along proximal section of the central feeding pipe 703. After positioning bushing 707 on the proximal section of shaft 701, the proximal edge of the central feeding pipe 703 is flanged with formation flange 713, and preferably also with application of a deformable o-ring 716, more preferably constructed from a cryogenically stable polymer, for sealing the gap between the proximal sections of the internal surface of bushing 707 and the central feeding pipe 703. A first through channel 714 communicates between the internal and external spaces of bushing 707, between its inner intermediate section 715 and its outer middle section 709.

A female unit 717 of the quick coupling preferably features housing 718 with a cylindrical inner cavity, wherein the diameter of the cylindrical section 719 of the inner cavity preferably conforms to the outer diameter of the distal and proximal sections 708 of bushing 707.

The cylindrical section 719 is optionally and preferably provided with distal and proximal annular grooves 720 and 721, for receiving sealing O-rings 723 and 722. Optionally more than two annular grooves are provided, with a corresponding number of O-rings.

A through opening 724 in the proximal face plane of the inner cavity serves for installation of an inlet connection 725 supplying the cryogen into cryoprobe 700. It should be noted, that the tolerances of the bushing and the housing preferably permit sliding insertion of bushing 707 of cryoprobe 700 into housing 718 of the female unit 717; the polymer o-rings 723 and 722 installed in the aforementioned annular grooves 720 and 721 of housing 718 ensure required sealing.

In addition, the inner surface of the face plane of housing 718 is preferably provided with blind holes 726, and helical springs 727 which are partially situated in these blind holes 726. In such a way, in the process of coupling, the male unit 710 of the coupling pair is spring-actuated by these helical springs 727.

A second through channel 728 with an outlet connection 729 installed on the outer end of the second through channel 728, communicates between the annular channel formed between the middle section 709 of bushing 707 and a space outside of housing 718. Thus, the first through channel 714, annular channel and second through channel 728 serve for exhausting evaporated cryogen from cryoprobe 700.

An electric heating spiral 820 is preferably situated between shaft 701 and the intermediate tube 704, as a non-limiting example of a heating element; this electric heating spiral 820 is energized by the wires via opening 825 in the shaft 701.

A quick electrical coupling 830 preferably comprises a male dielectric bushing 821 with opening 823 for wires of the electric heat spiral 820 and contacts 829 installed on the outer surface of this male dielectric bushing 821.

A corresponding female dielectric bushing 822 of the quick electrical coupling 830 is installed on distal face plane of the female unit 717 and preferably comprises internal contacts 824 and openings 827 for wires which supply electrical power from the external contacts 826.

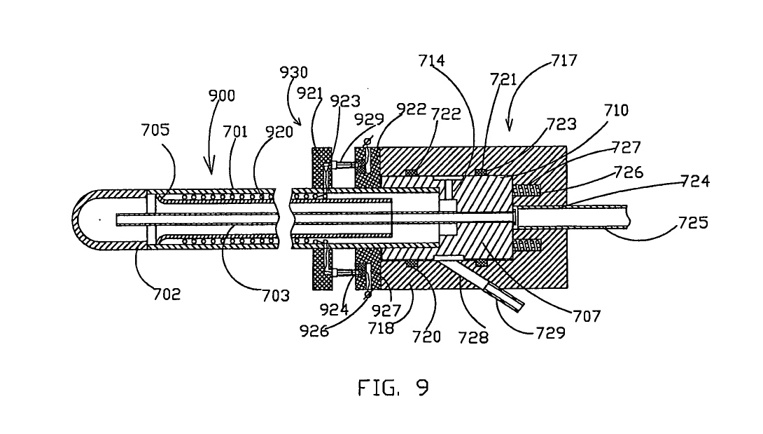

FIG. 9 is an axial cross-section of an optional embodiment of cryoprobe 900 constructed as it is shown in FIG. 7a, 7b and FIG. 7c; this cryoprobe is also preferably provided with the internal electric heating spiral and another electrical quick coupling unit. Reference numbers that are the same as for FIG. 7 or 8 have the same or similar function.

This embodiment, which features changes to cryoprobe 700, comprises hollow shaft 701, provided at its distal end with cryotip 702. Shaft 701 is preferably fabricated from a rigid material.

A central feeding pipe 703 is situated in shaft 703; the proximal end of the central feeding pipe 703 protrudes somewhat from the proximal end of shaft 701. The extreme proximal section of the central feeding pipe 703 is turned-along. The proximal sections of shaft 701 and the central feeding pipe 703 permit a male unit 710 of a quick coupling mechanism to be installed. The quick coupling mechanism is preferably constructed as previously described.

An electric heat helix 920 is situated between shaft 701 and the intermediate tube 704, and is an example of a heating element. Helix 920 is energized by the wires via opening 925 in the shaft 701.

A quick electrical coupling 930 comprises a male dielectric bushing 921 with opening 923 for wires of the electric heat spiral 920 and female sliding contacts 929 installed on a proximal face plane of the outer surface of this male dielectric bushing 921.

A female dielectric bushing 922 of the quick electrical coupling 930 is installed on distal face plane of the female unit 717, and comprises male sliding contacts 924 and openings 927 for wires which supplying electrical power from the external contacts 926.

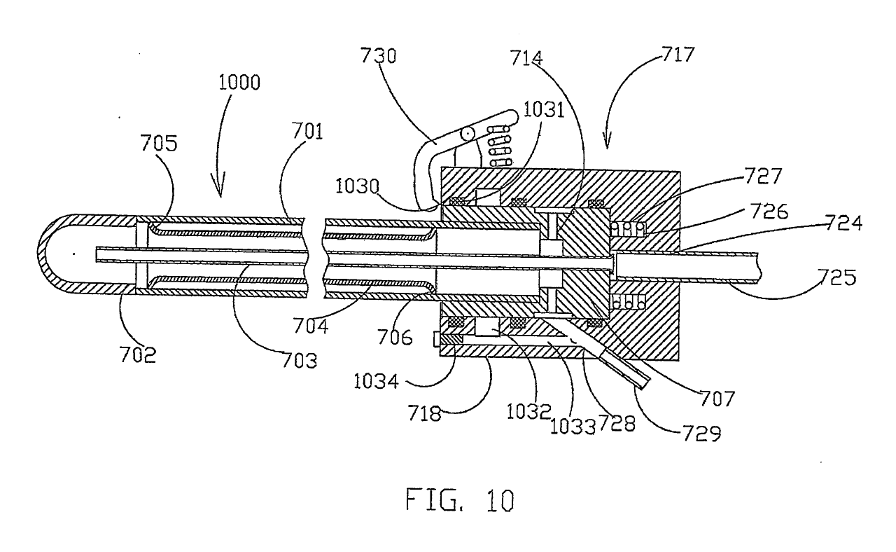

FIG. 10 shows an axial cross-section of an optional embodiment of a cryoprobe 1000, wherein the female unit is provided with a distal annular groove with an additional O-ring installed in it, and a circular groove 1032, which serves for drainage of a liquid cryogenic fluid. In this embodiment, housing 718 comprises a third distal groove 1030 with an O-ring 1031 installed in it. An opening 1033 is located in the distal face plane of housing 718; the axis of the opening 1033 is in parallel to the longitudinal axis of housing 718. This opening 1033 is in fluid communication with the circular groove 1032 and opening 728 for exhausting the cryogenic fluid. The distal end of opening 1033 is sealed with plug 1034.

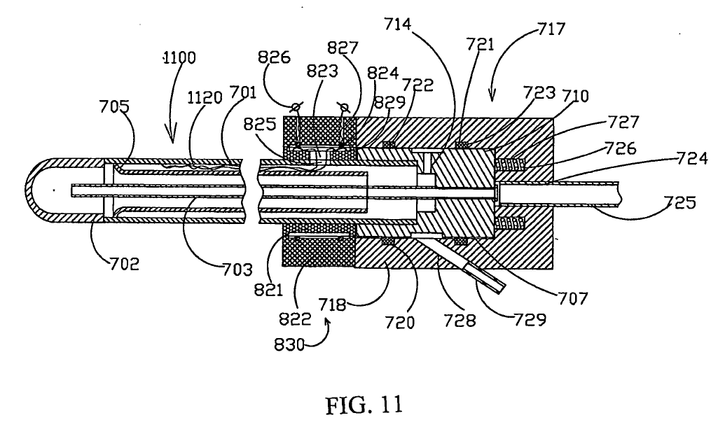

FIG. 11 is an axial cross-section of another optional embodiment of cryoprobe 1100 constructed as shown in FIG. 8, where a thermocouple is situated adjacently to the external shaft. This embodiment, featuring certain changes to cryoprobe 700 of FIG. 7, comprises hollow shaft 701, provided at its distal end with cryotip 702. Reference numbers that are the same as for FIG. 7 relate to the same or similar components. Shaft 701 is preferably fabricated from a rigid material.

A central feeding pipe 703 is situated in shaft 703. The proximal end of the central feeding pipe 703 protrudes somewhat from the proximal end of shaft 701. The extreme proximal section of the central feeding pipe 703 is turned-along. The proximal sections of shaft 701 and the central feeding pipe 703 provide for installation of a male unit 710 of a quick coupling mechanism.

Thermal insulation of shaft 701 is optionally and preferably ensured by an intermediate tube 704 with a flanged end 705, wherein the outer diameter of the formed flange 705 conforms to the internal diameter of the shaft. Friction between the internal surface of shaft 701 and flanging 705 ensures stable positioning of the intermediate tube 704 relative to shaft 701.

The male unit 710 of the quick coupling mechanism, which is installed on the proximal sections of shaft 701 and the central feeding pipe 703, comprises bushing 707. The outer surface of bushing 707 comprises proximal and distal cylindrical sections 708 and a middle section 709; the proximal and distal sections 708 have the same diameter, and diameter of the middle section 709 is preferably somewhat smaller.

The inner surface of bushing 707 preferably also has a stepped shape: it has distal, intermediate and proximal sections 711, 715 and 712, respectively, which preferably feature progressively decreasing diameters.

Bushing 707 is installed on the proximal sections of shaft 701 and the central feeding pipe 703 in such a manner that the distal section of the inner surface of the bushing is tightly fitted to the proximal section of the shaft and the proximal inner surface 712 of bushing 707 slidingly fits along on the turned-along proximal section of the central feeding pipe 703. After positioning bushing 707 on the proximal section of shaft 701, the proximal edge of the central feeding pipe 703 is flanged with formation flange 713, and preferably also with application of a deformable o-ring 716, more preferably constructed from a cryogenically stable polymer, for sealing the gap between the proximal sections of the internal surface of bushing 707 and the central feeding pipe 703. A first through channel 714 communicates between the internal and external spaces of bushing 707, between its inner intermediate section 715 and its outer middle section 709.

A female unit 717 of the quick coupling preferably features housing 718 with a cylindrical inner cavity, wherein the diameter of the cylindrical section 719 of the inner cavity preferably conforms to the outer diameter of the distal and proximal sections 708 of bushing 707.

The cylindrical section 719 is optionally and preferably provided with distal and proximal annular grooves 720 and 721, for receiving sealing O-rings 723 and 722. Optionally more than two annular grooves are provided, with a corresponding number of O-rings.

A through opening 724 in the proximal face plane of the inner cavity serves for installation of an inlet connection 725 supplying the cryogen into cryoprobe 700. It should be noted that the tolerances of the bushing and the housing preferably permit sliding insertion of bushing 707 of cryoprobe 700 into housing 718 of the female unit 717; the polymer o-rings 723 and 722 installed in the aforementioned annular grooves 720 and 721 of housing 718 ensure required sealing.

In addition, the inner surface of the face plane of housing 718 is preferably provided with blind holes 726, and helical springs 727 which are partially situated in these blind holes 726. In such a way, in the process of coupling, the male unit 710 of the coupling pair is spring-actuated by these helical springs 727.

A second through channel 728 with an outlet connection 729 installed on the outer end of the second through channel 728, communicates between the annular channel formed between the middle section 709 of bushing 707 and a space outside of housing 718. Thus, the first through channel 714, annular channel and second through channel 728 serve for exhausting evaporated cryogen from cryoprobe 700.

A thermocouple 1120 is preferably situated between shaft 701 and the intermediate tube 704, as a non-limiting example of a heating element; this thermocouple 1120 is connected by the wires via opening 825 in the shaft 701 with a control unit, which is not shown.

A quick electrical coupling 830 preferably comprises a male dielectric bushing 821 with opening 823 for wires of thermocouple 1120 and contacts 829 installed on the outer surface of this male dielectric bushing 821.

A corresponding female dielectric bushing 822 of the quick electrical coupling 830 is installed on distal face plane of the female unit 717 and preferably comprises internal contacts 824 and openings 827 for wires which supply electrical power from the external contacts 826.

Persons skilled in the art will appreciate that the present invention is not limited to what has been particularly shown and described hereinabove. Rather the scope of the present invention is defined by the appended claims and includes both combinations and sub combinations of the various features described hereinabove as well as variations and modifications thereof, which would occur to persons skilled in the art upon reading the foregoing description.

1. A cryosurgical instrument in fluid communication with a supply of liquid cryogen, said cryosurgical instrument comprising: an external shaft,
a central feeding pipe, at least partially located in said shaft, wherein the proximal end of said central feeding pipe protrudes at least partially from a proximal end of said external shaft;
a cryotip sealed with distal edge of said external shaft;
a male unit of a quick coupling mechanism comprising the proximal section of said external shaft;
an associated female unit of said quick coupling mechanism; said female unit having a form of a hollow cylinder sealed at its proximal end, wherein a proximal face plane of said cylinder is provided with an opening for said supply of said cryogen medium and wherein an internal surface of said hollow cylinder is provided with an annular groove for collection of exhausted cryogen medium from said cryosurgical instrument; said annular groove being in fluid communication with an exhaust outlet.

2. The cryosurgical instrument of claim 1, further comprising an external bushing installed on the proximal section of said external shaft for locking the proximal end of the external shaft.

3. The cryosurgical instrument of claim 2, wherein said bushing and/or said hollow cylinder are provided with at least two circular grooves and associated O-rings installed in said circular grooves.

4. The cryosurgical instrument of claim 3, further comprising:
an outer annular groove in the proximal section of said central feeding pipe;
an internal annular groove in an internal surface of said shaft opposing said outer annular groove of said central feeding pipe; and
an omega shaped flat spring, wherein at least lower sections of said omega shaped flat spring are positioned in said internal annular groove of said shaft, and middle sections of said omega shaped flat spring are positioned in said outer annular groove of said central feeding pipe.

5. The cryosurgical instrument of claim 4, further comprising a second bushing on the proximal section of said central feeding pipe.

6. The cryosurgical instrument of claim 5, wherein said shaft comprises a rigid material.

7. The cryosurgical instrument of claim 6, wherein said male unit further comprises an intermediate tube with two flanged ends for thermal insulation of said shaft, wherein outer diameters of said flanged ends conform to an internal diameter of said shaft.

8. The cryosurgical instrument of claim 7, wherein the first and second bushings comprise a polymer.

9. The cryosurgical instrument of claim 8, further comprising a spring-actuated ratchet on an outer surface of said cylindrical housing for locking said quick coupling mechanism.

10. The cryosurgical instrument of claim 9, wherein said male unit further comprises an external tube with two ends having inward flanges for thermal insulation of said shaft, and wherein inner diameters of said flanges conform to an outer diameter of said shaft.

11. The cryosurgical instrument of claim 10, wherein the male unit further comprises an intermediate tube, and the shaft further comprises two annular hollows having an internal diameter corresponding to an outer diameter of the intermediate tube, wherein friction between said annular hollows and said intermediate tube ensures a stable positioning of said intermediate tube in an internal cavity of the shaft.

12. A cryoprobe in fluid communication with a supply of liquid cryogen, the cryoprobe comprising:
a quick coupling mechanism, said quick coupling mechanism comprising a male unit and a female unit, wherein the male unit comprises:
a hollow shaft, wherein a proximal section of said shaft has a smaller outer diameter than middle and distal sections of the shaft;
a central feeding pipe, at least partially located in said shaft, wherein a proximal end of said central feeding pipe protrudes at least partially from a proximal end of said shaft and wherein a proximal section of said central feeding pipe has a reduced diameter;
a bushing, wherein an outer surface and an internal surface of said bushing are stepped and wherein said outer surface of said bushing comprises a proximal cylindrical section and a distal cylindrical section; an outer diameter of said distal cylindrical section being larger than an outer diameter of said proximal cylindrical section; and wherein an inner surface of said bushing comprises a distal section, an intermediate section and a proximal section with progressively decreasing diameters; said bushing being installed on the proximal section of said shaft and on the proximal section of the central feeding pipe such that the distal section of the inner surface of said bushing is fitted on the proximal section of said shaft and the proximal section of the inner surface of said bushing is fitted slidingly on the proximal section of said central feeding pipe; and
the female unit of said quick coupling mechanism comprises a housing for receiving at least a portion of said male unit, said female unit comprising a stepped cylindrical inner cavity, wherein a diameter of a distal section of said inner cavity conforms to the outer diameter of the distal section of said bushing, and a diameter of a middle section of said inner cavity conforms to the outer diameter of the proximal section of said bushing; wherein the proximal section of said inner cavity receives a supply of cryogen into said cryoprobe; the distal and proximal sections of the outer surface of said bushing and the distal and intermediate sections of the inner cavity of said housing form an annular channel between surfaces of the proximal section of the outer surface of said bushing and the distal section of the inner cavity of said housing.

13. The cryosurgical instrument of claim 12, wherein said female unit further comprises a second through channel with an outlet connection on an outer end of said second through channel; said second through channel communicates between said annular channel and a space outside of said housing.

14. The cryosurgical instrument of claim 13, wherein a proximal edge of said central feeding pipe is flanged with a deformable o-ring, and further comprising a first through channel communicating between internal and external spaces of said bushing.

15. The cryosurgical instrument of claim 14, further comprising an intermediate tube with two flanged ends for at least partially surrounding and insulating said shaft, wherein an outer diameter of said flanged ends conforms to an internal diameter of said shaft.

16. The cryosurgical instrument of claim 15, further comprising a spring-actuated ratchet, on the outer surface of said housing, for locking said quick coupling mechanism.

17. The cryosurgical instrument of claim 16, wherein an inner surface of a face plane of the housing comprises a plurality of blind holes, and helical springs are partially situated in the blind holes, for locking said quick coupling mechanism.

18. The cryosurgical instrument of claim 12, further comprising an external tube with two ends having inward flanges, and wherein an inner diameter of said flanges conforms to an outer diameter of said shaft; wherein friction between an outer surface of said shaft and said flanges of said external tube ensures stable positioning of said external tube with regard to said shaft.

19. The cryosurgical instrument of claim 15, wherein the male unit further comprises an intermediate tube, and the shaft further comprises a plurality of annular hollows having an internal diameter corresponding to an outer diameter of the intermediate tube, wherein friction between said annular hollows and said intermediate tube ensures stable positioning of said intermediate tube in an internal cavity of the shaft.

20. The cryosurgical instrument of claim 16, wherein an internal cylindrical section of the housing of said female unit comprises a plurality of distal and proximal annular grooves, for receiving a plurality of sealing O-rings.

21. A cryoprobe comprising a quick coupling mechanism, said quick coupling mechanism comprising a male unit and a corresponding female unit, the cryoprobe further comprising a hollow shaft and an intermediate tube, each of which is capable of being dissembled and each of which, upon assembly, is partially disposed within said male unit and partially within said female unit; the cryoprobe further comprising an electric heating element, which is situated between the shaft and the intermediate tube, and electric wires for energizing said electric heating element, said wires entering through an opening in said shaft; wherein said quick coupling mechanism further comprises a quick electrical coupling, wherein male and female units of said quick electrical coupling comprise a male dielectric bushing with an opening for wires of said electric heating element and contacts installed on said male dielectric bushing, and a female dielectric bushing with opposite contacts.

22. The cryosurgical instrument of claim 21, wherein the cryoprobe further comprises a control unit and one or more thermal sensors arranged in internal space, and the quick electrical coupling serves for transferring a signal from said thermal sensors to said control unit.

23. The cryosurgical instrument of claim 12, wherein an internal cylindrical section of the housing of said female unit comprises first and second annular grooves, for receiving sealing O-rings, a circular groove for drainage of a liquid cryogenic fluid and a third distal groove for receiving an O-ring.

24. The cryosurgical instrument of claim 23, wherein a distal face of said housing further comprises an opening in fluid communication with said circular groove for exhausting cryogenic fluid and a plug for blocking said opening.

25. A cryoprobe in fluid communication with a supply of liquid cryogen, the cryoprobe comprising:
a quick coupling mechanism, said quick coupling mechanism comprising a male unit and a female unit, wherein the male unit comprises:
a hollow shaft, wherein a proximal section of said shaft has a smaller outer diameter than middle and distal sections of the shaft;
an external bushing installed on the proximal section of said shaft for locking the proximal end of the external shaft;
a central feeding pipe, at least partially located in said shaft, wherein the proximal end of said central feeding pipe protrudes at least partially from a proximal end of said shaft;
an outer annular groove in the proximal section of said central feeding pipe;
an internal annular groove in an internal surface of said shaft opposing said outer annular groove of said central feeding pipe;
an omega shaped flat spring, wherein at least lower sections of said omega shaped flat spring are positioned in said internal annular groove of said shaft, and middle sections of said omega shaped flat spring are positioned in said outer annular groove of said central feeding pipe;
a second bushing on the proximal section of said central feeding pipe; and
a cryotip, joined to said shaft at a distal edge;
and wherein the female unit of said quick coupling is in fluid communication with the supply of liquid cryogen, and comprises:
a cylindrical housing with an exhausting opening in a wall of the housing for removal of evaporated cryogen; a proximal face plane of said housing comprising a central opening for delivery of said liquid cryogen, and being shaped for contacting said second bushing of said central feeding pipe; and
a spring-actuated metal ring, displaceable along an internal wall of said cylindrical housing for contacting said first bushing of said shaft.
